(19) United States
(12) Patent Application Publication
(10) Pub. No.: US 2010/0074975 A1
(43) Pub. Date: Mar. 25, 2010

(54) Pharmaceutical composition for the prevention and treatment of liver disease comprising a lonicera caerulea L. Var. Edulis extract

(76) Inventors: Ju Hwan Eum, Seoul (KR); Man Chul Suh, Seoul (KR); Dur Han Kwon, Daejeon (KR); Byeong ku Yoon, Chungcheongbuk-do (KR); Wha jeong Choi, Daejeon (KR); Kyung mi Kwon, Daejeon (KR); Chan soo Kim, Jeju-do (KR)

Correspondence Address: John W Renner;Renner Otto Boisseller & Sklar, 1621 Euclid Avenue 19th Floor, Cleveland, OH, 44115, US

(21) Appl. No.: 11/988,704

(22) PCT Filed: Jul. 7, 2006
(86) PCT No.: PCT/KR2006/063026

§ 371 Date: Jan. 10, 2008

(30) Foreign Application Priority Data
Jul. 13, 2005 (KR) 10-2005-0063026

(51) Int. Cl. A61K 36/355; A61P 1/16

(52) U.S. Cl. 424/725

(57) ABSTRACT

Disclosed herein is a pharmaceutical composition having preventative and therapeutic effects on liver diseases, comprising an extract from Lonicera caerulea L. var. edulis. The composition has preventative and therapeutic effects on hepatitis, liver cirrhosis, fatty liver, and the like.

### TECHNICAL FIELD

The present invention relates to a pharmaceutical composition having preventative and therapeutic effects on liver diseases, comprising an extract from Lonicera caerulea L. var. edulis.

### BACKGROUND ART

The liver, situated between the digestive system and the systemic circulatory system, plays important roles in protecting the whole body from foreign toxic substances and in the metabolism of exogenous materials. Since the exogenous materials taken up by the body initially enter the liver to be filtered, the liver has a high risk of being exposed to numerous toxic substances as well as nutrients. Thus, the liver is highly vulnerable to damage relative to other organs.

Liver diseases are classified into two major types according to cause: one is toxic liver disease caused by the excessive ingestion of alcohol or the like, and the other is viral liver disease caused by viral infection. Viral liver diseases arise from infection with hepatitis B virus, hepatitis C virus, or the like. Recently, toxic liver disease is increasing due to food, medicaments, medicinal herbal substances, alcohol, and the like. Liver diseases are difficult to diagnose in early stages due to the absence of subjective symptoms. By the time individuals develop subjective symptoms, the liver has suffered great damage. The liver is an organ which has greater ability to recover its full function than other organs. However, it is difficult to restore normal liver function when hepatocytes have already been transformed.

To date, potentially therapeutic agents for treating chronic hepatitis or liver cirrhosis have not been developed. Interferons and lamivudine, which is a nucleic acid analogue, have been used to treat chronic hepatitis. However, these drugs exert the effect of inhibiting viral activity but do not restore the normal function of hepatocytes. Silymarin, which is known as an agent for restoring the function of hepatocytes, is most commonly used worldwide. Silymarin is extracted from a medicinal herb, Carduus marianus Linne Silybum marianum, which has been known as an important medicinal herb since Before Christ (B.C.) in Western countries, including ancient Greece. Since silymarin has been domestically introduced as a therapeutic agent for liver damage in the 1970's, many pharmaceutical preparations containing it as a major ingredient have been developed and are now available on the market. Pharmaceutical preparations containing silymarin as a major ingredient have already been widely applied for the clinical purpose of treating liver diseases. However, the major ingredient silymarin has a drawback in that it is not highly water-soluble, and thus has a low uptake in the body when orally administered. At present, silymarin preparations have been used merely in auxiliary therapy for liver diseases, such as toxic liver diseases, chronic hepatitis and liver cirrhosis. Thus, there is a need for the development of drugs capable of rapidly restoring the normal function of hepatocytes.

Many studies have been performed to determine whether natural substances have effects of improving liver function. For example, Korean Pat. Registration No. 80759 discloses fermented milk, which is useful for maintaining and improving liver function, and a method of preparing the same. Korean Pat. Laid-open Publication No. 2003-0027615 discloses a functional food composition containing an extract from fruits of Hovenia dulcis Thunb, the composition having effects of enhancing liver function and relieving hangover symptoms. Korean Pat. Laid-open Publication No. 2003-0011818 discloses the use of an extract from Eleutherococcus senticosus in the production of functional rice coated therewith. Korean Pat. Laid-open Publication No. 2003-0063308 discloses a therapeutic agent for hepatitis B comprising an extract from the medicinal herb Phyllanthus urinaria, and a method of preparing the same. Korean Pat. Laid-open Publication No. 2004-0018733 discloses a composition for treating viral liver diseases comprising an extract from Ixeris sonchifolia.

Based on this background, the present inventors conducted intensive and thorough research to obtain from natural materials a substance having good therapeutic activity against liver diseases, other than conventional therapeutic compositions as described above. The research resulted in the finding that when a damaged hepatic cell line was dosed with an extract from Lonicera caerulea L. var. edulis, cell growth was stimulated, bringing about the restoration of damaged liver function, which was determined by remarkable decreases in GOT and GPT levels as biochemical markers for liver function. These results revealed that a composition comprising the extract from Lonicera caerulea L. var. edulis has good preventive and therapeutic effects on liver diseases, such as hepatitis, liver cirrhosis and fatty liver, thereby leading to the present invention.

### DISCLOSURE

### Technical Problem

Accordingly, the present invention aims to provide a pharmaceutical composition having preventative and therapeutic effects on liver diseases, comprising an extract from Lonicera caerulea L. var. edulis.

### DESCRIPTION OF DRAWINGS

FIG. 1 shows the effects of an extract from fruits of Lonicera caerulea L. var. edulis on the growth of HepG2 cells.

FIG. 2 shows the effects of ethyl alcohol on the growth of HepG2 cells.

FIG. 3 shows the comparison of absorbance of a well treated with ethyl alcohol alone and wells treated with ethyl alcohol and then an extract from fruits of Lonicera caerulea L. var. edulis with that of a control well not treated with either ethyl alcohol or the extract from fruits of Lonicera caerulea L. var. edulis.

FIG. 4 shows the restoration of liver function by administration of an extract from fruits of Lonicera caerulea L. var. edulis, which was observed on a SPOTCHEM™ II strip.

FIG. 5 shows the results of histochemical analysis on the effects of an extract from fruits of Lonicera caerulea L. var. edulis and silymarin on acute hepatitis when they were administered into a transgenic mouse model of acute hepatitis.

### BEST MODE

In one aspect, the present invention relates to a pharmaceutical composition having preventative and therapeutic effects on liver diseases, comprising an extract from Lonicera caerulea L. var. edulis.

The term “extract”, as used herein, refers to an active ingredient isolated from a natural material. In the present invention, the extract may be obtained by an extraction process using water, an organic solvent, or a solvent mixture thereof, and includes dry powder of the extract or all forms formulated therefrom.

The term “Lonicera caerulea L. var. edulis”, as used herein, refers to all organs, for example, roots, branches, stems, leaves, flowers and fruits, of natural, hybrid or variant types of Lonicera caerulea L. var. edulis, but preferably indicates fruits of Lonicera caerulea L. var. edulis. Lonicera caerulea L. var. edulis is a dicotyledonous plant belonging to the Family Caprifoliaceae of the Order Rubiales. It is a deciduous shrub that grows to 1.5 m tall, is densely branched, and has shield-shaped bracts at nodes of twigs. The inner part of branches is white. The leaves are opposite, lanciform or elliptic and blunt- or sharp-ended, lack teeth on the margins, have short hairs on the margins and surface, and have many wooly hairs underneath. The flowers usually have short stalks, which arise from leaf axils, have trumpet-shaped creamy white corollas, and bloom in summer. Each calyx contains five toothed sepals. The corollas are yellowish white, cylindrical campanulate, 1.2-1.5 cm long, and slightly hairy. The stamens are shorter than styles and have no hairs, and the two ovaries are fused together. The fruits are oval or nearly circular, ripen to purplish black between July and October, and are covered with white powder. This deciduous shrub is an arctic plant that is widespread in Siberia, Sakhalin, the Northern region of China, Tibet, North Korea, and the like. Lonicera caerulea L. var. edulis was not investigated prior to the present invention for effects of stimulating the growth of hepatocytes and improving liver function due to the stimulatory effect.

The effect of improving liver function can be objectively evaluated by measuring the degree of stimulation of growth rates of damaged hepatocytes due to composition administration and levels of hepatic enzymes, aspartate aminotransferase (AST, also known as GOT) and alanine aminotransferase (ALT, also known as GPT). ALT and AST are enzymes present in hepatocytes. When hepatocytes are damaged or disrupted, these enzymes are released therefrom, leading to an increase in concentrations thereof in the blood. Thus, ALT and AST levels are used as biochemical indicators for liver diseases caused by liver cell damage.

In a detailed practice of the prevent invention, a human liver cell line, HepG2, was damaged with ethyl alcohol and then dosed with an extract from Lonicera caerulea L. var. edulis, and stimulated growth rates were compared with those of a control not dosed with the extract from Lonicera caerulea L. var. edulis (also referred herein to simply as “L. caerulea extract”). The L. caerulea extract was found to stimulate the growth of damaged hepatocytes in a dose-dependent manner. The L. caerulea extract stimulated the cell growth by about 27% at 0.25 mg/ml and about 54% at 0.5 mg/ml. In addition, ALT and AST levels were measured to determine whether liver function was enhanced. The administration of L. caerulea extract resulted in a reduction of ALT and AST levels in HepG2 cells. When the L. caerulea extract was administered into a transgenic mouse of acute hepatitis, it exhibited greater ability to restore liver function by 25% more than the conventional drug silymarin. These results demonstrate that the L. caerulea extract of the present invention has an effect of enhancing liver function by stimulating the growth of hepatocytes and thus has therapeutic activity against liver diseases.

The term “liver diseases”, as used herein, refers to all diseases that bring about decreased liver function. Liver diseases are caused by viruses (e.g., hepatitis virus A, B, C, D or E), alcohol, drugs (antituberculosis drugs, aspirin, antibiotics, anesthetics, antihypertensive drugs, oral contraceptives, etc.), congenital metabolic disorders, and the like. Detailed examples of liver diseases are liver hepatitis, liver cirrhosis and fatty liver. Liver hepatitis includes chronic and acute liver hepatitis.

The term “prevention”, as used herein, refers to all actions that inhibit or delay the reduction of liver function through composition administration. The term “treatment”, as used herein, refers to all actions that restore or beneficially change liver function and liver regeneration through composition administration.

The administration of the present composition may prevent and treat liver hepatitis, liver cirrhosis, fatty liver, and other liver diseases, as well as symptoms or complications caused by the diseases. Examples of symptoms of liver diseases, which can be prevented and treated by the L. caerulea extract, include fatigue, vomiting, decreased appetite, abdominal pain, and jaundice. Examples of complications include edema, ascites, gastrointestinal bleeding, esophageal variceal bleeding, and hepatic encephalopathy (hepatic coma).

In the present invention, the L. caerulea extract is prepared using extraction with water, an organic solvent, or a solvent mixture thereof. The resulting extract may be used as it is or after being concentrated and/or dried.

When an organic solvent is used, the extraction process is carried out at room temperature or by heat treatment under conditions that prevent the destruction of effective ingredients or minimize such destruction using an organic solvent, such as methanol, ethanol, isopropanol, butanol, ethylene, acetone, hexane, ether, chloroform, ethylacetate, butylacetate, dichloromethane, N,N-dimethylformamide (DMF), dimethylsulfoxide (DMSO), 1,3-butylene glycol, propylene glycol, or a solvent mixture thereof. Since the degree of extraction and loss of an effective ingredient may vary depending on the organic solvent used, a suitable organic solvent must be selected and employed. The extraction method is not specifically limited, and includes cold precipitation, ultrasonic extraction, and reflux extraction.

The solvent extraction may further include a step of filtering the extract to remove suspending solid particles. The removal of particles may be achieved using cotton, nylon, and the like, or using ultrafiltration, freezing filtration, centrifugation, and the like, but the present invention is not limited to the examples.

The concentration of the extract may be performed using reduced pressure, reverse osmosis, and the like. The concentrate is dried by freeze drying, vacuum drying, hot wind drying, spray drying, drying under reduced pressure, foam drying, high frequency drying, infrared drying, and the like, but the present invention is not limited to the examples. If desired, the present method may further include a step of pulverizing the final dried extract.

In addition, the extract may be optionally subjected to a fractionation process. For example, the extract is suspended in distilled water, and extracted using a nonpolar organic solvent, such as hexane, ether, dichloromethane, chloroform, ethylacetate, or a solvent mixture thereof to separate a nonpolar solvent-soluble layer. The obtained nonpolar solvent-soluble layer is concentrated and/or dried.

In a detailed practice, the L. caerulea extract of the present invention was obtained by hot water extraction, cold water extraction, ultrasonic extraction or reflux extraction, preferably reflux extraction, using water, C₁ to C₄ lower alcohol or a solvent mixture thereof weighing 5 to 25 times, preferably 7 to 15 times as much as the dry weight (kg) of Lonicera caerulea L. var. edulis, preferably fruits thereof. The extraction was carried out at 20° C. to 100° C., preferably 60° C. to 100° C., for a period ranging from 0.5 hrs to 2 days, preferably 1 hr to 1 day, and was serially performed 1-5 times, preferably 2-3 times. The extract was passed through filter paper. The filtrate was concentrated under reduced pressure using a rotary vacuum concentrator at 20° C. to 100° C., preferably 50° C. to 70° C., and dried, thereby yielding the L. caerulea extract in powder form according to the present invention. The L. caerulea extract in the powder form may be used as it is or after being dissolved in a solvent at a predetermined concentration.

The L. caerulea extract is safe and does not cause side effects or stimulate resistance thereto because it contains substances obtained from natural material as effective ingredients. Thus, the L. caerulea extract has an advantage in that it is able to be administered for a long period of time. Actually, an acute toxicity test in mice revealed that the L. caerulea extract is not toxic. Also, the composition may be applied to humans, as well as livestock including cattle, horses, sheep, pigs, goats, antelopes and dogs.

The L. caerulea extract is contained in the pharmaceutical composition for preventing and treating liver diseases in an amount of 0.01% to 100%, and more preferably 1% to 80% by weight based on the total weight of the composition. Also, the composition may further include an additive which does not increase efficacy but is commonly used in pharmaceutical compositions to enhance flavor, taste, color, or the like. The composition may further include inorganic and organic additives, such as vitamins B₁, B₂, B₆, C and E, niacin, carnitine, betaine, folic acid, pantothenic acid, biotin, zinc, iron, calcium, chrome, magnesium, and mixtures thereof. The composition may be used alone, or may further include a conventionally used substance having therapeutic activity against liver diseases.

The composition may include a pharmaceutically acceptable carrier, and may be formulated into dosage forms for human or veterinary use. According to the intended use, the composition may be formulated into a variety of ordinary forms suitable for oral, parenteral and topical administration. Oral solid preparations, such as powders, granules, tablets and capsules, may be prepared using binders, lubricants, integrators, excipients, solubilizers, dispersing agents, stabilizers, suspending agents, pigments, flavors, and the like. Oral liquid preparations, such as suspensions, solutions, emulsions and syrups, may be prepared with commonly used simple diluents, water and liquid paraffin, as well as humectants, sweeteners, aromatics, preservatives, and the like. Injectable preparations may be prepared by mixing buffers, preservatives, analgesics, solubilizers, isotonic agents, stabilizers, and the like. Such a composition may be presented in unit-dose (single dose) or multiple dose (several doses) containers, for example, sealed ampoules and vials, and may be stored in a freeze-dried (lyophilized) condition requiring only the addition of a sterile liquid carrier, for example water for injections, immediately prior to use. Extemporaneous injection solutions and suspensions may be prepared from sterile powders, granules and tablets.

In another aspect, the present invention relates to a method of preventing and treating liver diseases, comprising administering a composition comprising the L. caerulea extract to a patient.

The term “patient”, as used herein, refers to a human or an animal, such as horses, sheep, pigs, goats, camels, antelopes and dogs, which has a disease characterized by reduced or damaged liver function, for example, hepatitis, liver cirrhosis, or fatty liver, and symptoms of the disease may be relieved through the administration of the present composition. The aforementioned diseases may be effectively prevented and treated by administering the composition comprising the L. caerulea extract of the present invention to a patient. The present composition may be administered in combination with a conventional therapeutic agent for liver diseases.

The term “administration”, as used herein, refers to the introduction of a predetermined material into a patient using any suitable method. The present composition may be orally or parenterally administered via any of the common routes, as long as it is able to reach the desired tissue. Also, the composition may be administered using a certain apparatus capable of transporting active substances into target cells.

The present composition may be administered in a pharmaceutically effective amount.

The term “pharmaceutically effective amount”, as used herein, refers to an amount sufficient for treating or preventing diseases, which is commensurate with a reasonable benefit/risk ratio applicable for medical treatment or prevention. An effective dosage amount of the composition may be determined depending on the patient's gender and age, the severity of the illness, drug activity, sensitivity to drugs, administration time, administration routes and excretion rates, treatment duration, drugs used in combination with the composition; and other factors known in medicine. The present composition may be administered as a sole therapeutic agent or in combination with other therapeutic agents, and may be administered sequentially or simultaneously with conventional therapeutic agents. This administration may be provided in single or multiple doses. Taking all factors into consideration, it is important to conduct administration with the minimum of doses that is capable of realizing the greatest effects with no adverse effects, such doses being readily determined by those skilled in the art. The extract prepared according to the preparation method of the present invention is preferably administered orally or intravenously. In general, a single dose of the extract is preferably 1 to 20 mg/kg adult for oral administration, and preferably 1 to 20 mg/kg adult for intravenous administration. The dosage for a specific patient may vary depending on the patient's gender, age, health state and diet, administration time, administration modes, co-administered drugs, and severity of illness.

### MODE FOR INVENTION

A better understanding of the present invention may be obtained through the following examples which are set forth to illustrate, but are not to be construed as the limit of the present invention.

### Example 1

### Preparation of an Extract from Fruits of Lonicera caerulea L. var. edulis

(1-1) Hot Water Extraction Using Water as a Solvent

Fruits of native Lonicera caerulea L. var. edulis were directly collected in Yanbian of China and Baekdu Mountain, and dried for use in experiments. 100 g of pulverized L. caerulea fruits were added to 1 liter of distilled water and agitated. The resulting solution was extracted under reflux for 3 hrs at 90° C. to 95° C. and filtered. The obtained galenic extract was concentrated under reduced pressure at 55° C. to 65° C. and freeze-dried, thereby yielding 21.2 g of a galenic composition powder extract.

(1-2) Hot Water Extraction Using a Solvent Mixture of Water and Alcohol

1 liter of 25% ethyl alcohol was added to 100 g of pulverized L. caerulea fruits as used in Example 1-1, and agitated. Then, the resulting solution was extracted under reflux for 3 hrs at 80° C. to 90° C. and filtered. The obtained galenic extract was concentrated under reduced pressure at 55° C. to 65° C. and freeze-dried, thereby yielding 19.5 g of a galenic composition powder extract.

### Example 2

### Evaluation of the Effect of the Extract from Fruits of Lonicera caerulea L. var. edulis on the Growth of Hepatocytes

HepG2 cells were seeded in a 96-well plate at a density of 1×10⁴ cells per well, and incubated in a culture medium supplemented with 10% fetal bovine serum (FBS) for 16 hrs. After the 96-well plate was washed with physiological saline, the extract from L. caerulea fruits, which was diluted in culture medium in amounts of 0, 0.125, 0.25, 0.5 and 1 mg/ml, was added to each well. After incubation for 48 hrs, the cell number in each well was measured using a SRB method.

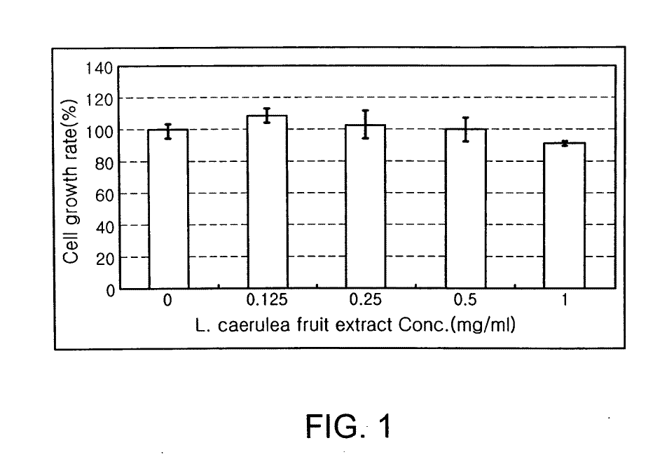

After the incubation for 48 hrs, the culture medium containing the extract from L. caerulea fruits was removed from the 96-well plate. The 96-well plate was washed with physiological saline, and the cells in each well were fixed with 70% acetone for 20 min. The fixed cells were dried, stained with a SRB solution for 30 min, and washed with a 1% acetic acid solution five times to eliminate unbound SRB. After the cells were dried again, 10 mM Tris was added to the cells to dissolve cellular proteins and unbound SRB, and absorbance was measured at 562 nm using a spectrophotometer. The results were expressed as a percentage by comparing the absorbance of each well treated with the extract from L. caerulea fruits with that of a control well not treated with the extract from L. caerulea fruits (FIG. 1). When cells were dosed with the extract from L. caerulea fruits at up to 0.5 mg/ml, their number stayed the same or increased slightly, or their growth was little affected in comparison with the well not treated with the extract from L. caerulea fruits.

### Example 3

### Evaluation of the Effect of Ethyl Alcohol on the Growth of Hepatocytes

HepG2 cells were seeded in a 96-well plate at a density of 1×10⁴ cells per well and incubated in a culture medium supplemented with 10% FBS for 16 hrs. After the 96-well plate was washed with physiological saline, ethyl alcohol, which was diluted in a culture medium in amounts of 0, 0.5, 1.0, 1.5 and 2.0% (v/v), was added to each well in which HepG2 cells were cultured. After incubation for 48 hrs, the cell number in each well was measured using a SRB method.

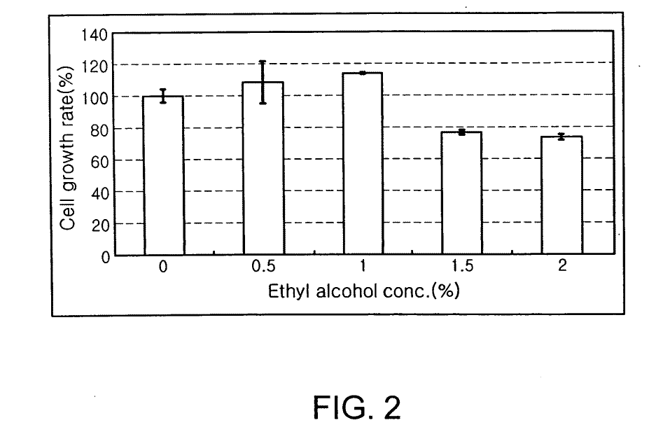

The culture medium was removed, and the 96-well plate was washed with physiological saline. The cells in each well were then fixed with 70% acetone for 20 min. The fixed cells were dried, stained with an SRB solution for 30 min, and washed with a 1% acetic acid solution five times to eliminate unbound SRB. After the cells were dried again, 10 mM Tris was added to the cells to dissolve cellular proteins and unbound SRB, and absorbance was measured at 562 nm using a spectrophotometer. The results were expressed as a percentage by comparing the absorbance of each well treated with ethyl alcohol with that of a control well not treated with ethyl alcohol (FIG. 2). Ethyl alcohol was found to stimulate cell growth at concentrations of up to 1%, but reduced cell number at concentrations of 1.5% or higher.

### Example 4

### Evaluation of the Ability of the Extract from L. caerulea Fruits to Restore the Growth of Cells Whose Growth is Suppressed by Ethyl Alcohol

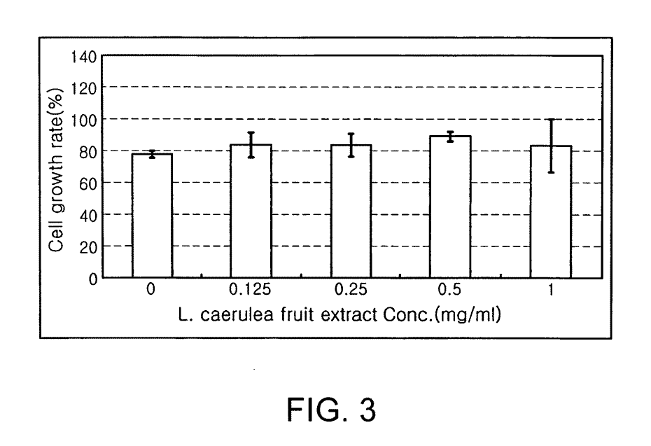

HepG2 cells were seeded in a 96-well plate at a density of 1×10⁴ cells per well, and incubated in a culture medium supplemented with 10% FBS for 16 hrs. After the 96-well plate was washed with physiological saline, ethyl alcohol, which was diluted in culture medium in 1.5% (v/v), was added to each well in which HepG2 cells were cultured. Cell damage was induced using ethyl alcohol for 24 hrs. Thereafter, the culture medium containing ethyl alcohol was removed, and the extract from L. caerulea fruits, which was diluted in culture medium in amounts of 0, 0.125, 0.25, 0.5 and 1 mg/ml, was added to each well. After incubation for 48 hrs, the culture medium was removed, and the 96-well plate was washed with physiological saline. Cells in each well were then fixed with 70% acetone for 20 min. The fixed cells were dried, stained with an SRB solution for 30 min, and washed with a 1% acetic acid solution five times to eliminate unbound SRB. After the cells were dried again, 10 mM Tris was added to the cells to dissolve cellular proteins and unbound SRB, and absorbance was measured at 562 nm using a spectrophotometer. The absorbance of a well treated with ethyl alcohol alone and the absorbance of each well treated with alcohol plus the extract from L. caerulea fruits were compared with that of a control well which was not treated with ethyl alcohol or with the extract from L. caerulea fruits (FIG. 3).

The well treated with 1.5% ethyl alcohol showed absorbance reduced by 22% in comparison with the well not treated with ethyl alcohol. The reduced absorbance increased in a manner that was dependent on the concentrations of the extract from L. caerulea fruits when the extract was administered at up to 0.5 mg/ml to the well treated with ethyl alcohol. These results indicate that the ethyl alcohol-induced cell growth damage was restored by administration of the extract from L. caerulea fruits.

Thus, the degree of restoration of ethyl alcohol-induced cell growth damage by administration of the extract from L. caerulea fruits was calculated according to Equation 1, and the results are given in Table 1, below.

Restoration rate of cell growth=(group dosed with the extract from L. caerulea fruits−group dosed with ethyl alcohol alone)/(non treatment group−group dosed with ethyl alcohol alone) [Equation 1]

TABLE 1
The ability of the extract from L. caerulea fruits to restore
the growth of cells whose growth is suppressed by ethyl alcohol

| Test groups | | Percentage (%) |
|---|---|---|
| Group not treated with ethyl alcohol or | | 100.00 ± 4.42 |
| with the extract from L. caerulea fruits | | |
| Group treated with ethyl alcohol alone | | 0.00 ± 1.96 |
| Group treated with ethyl alcohol | 0.125 mg/ml | 20.45 ± 7.72 |
| plus the extract from L. caerulea | 0.25 mg/ml | 27.14 ± 8.14 |
| fruits | 0.5 mg/ml | 54.77 ± 3.37 |

As shown in Table 1, in cells in which cell damage was induced by ethyl alcohol (1.5%), the extract from L. caerulea fruits restored cell damage by 20.45±7.72% at 0.125 mg/ml, 27.14±8.14% at 0.25 mg/ml, and 54.77±3.37 at 0.5 mg/ml. These results indicate that the extract from L. caerulea fruits acts as a medicinal agent restoring the function of liver tissue by stimulating the growth of damaged hepatocytes.

### Example 5

### Evaluation of the Ability of the Extract from L. caerulea Fruits to Restore Liver Function in Hepatocytes Damaged by Ethyl Alcohol

Hep3B cells were seeded in a 96-well plate at a density of 2×10⁴ cells per well, and were incubated for 12 hrs to allow them to adhere to the bottom of the plate.

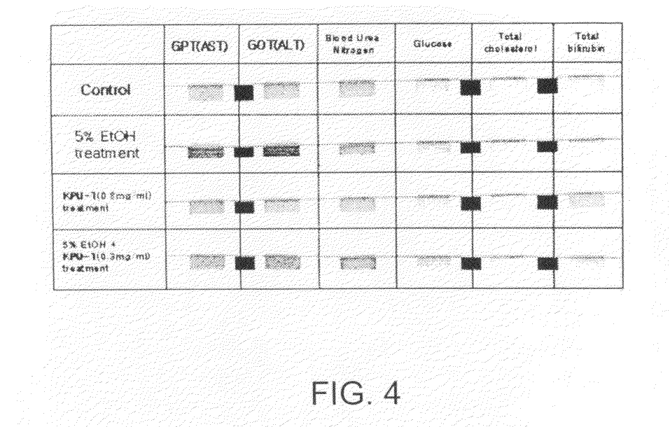

After the culture supernatant was removed, the plate was washed with physiological saline. 5% Ethyl alcohol in culture medium was added to each well, and incubated for 12 hrs to induced cell damage. After the culture supernatant was removed from each well, a culture medium containing the extract from L. caerulea fruits (0.3 mg/ml) and a culture medium not containing the extract were individually added to the well in which cell damage was induced by ethyl alcohol. ALT and AST levels were measured in wells treated with ethyl alcohol or not using a SPOTCHEM™ II strip from the Array Company in Japan. The SPOTCHEM™ II strip enables the measurement on a single strip of all of amounts of ALT (GOT), AST (GPT), blood urea nitrogen, glucose, total cholesterol and total bilirubin. The region measuring ALT and AST levels is present as a yellowish white zone in the strip. Increased ALT and AST levels in a sample change the yellowish white zone to dark blue. After 48 hrs, the culture supernatant of each well was collected and loaded onto the SPOTCHEM™ II strip, and the strip was observed for color change in the yellowish white zone (FIG. 4).

In FIG. 4, the control was a culture supernatant of Hep3B cells not treated with ethyl alcohol or with the extract from L. caerulea fruits. In the control, the yellowish white zone was changed to light blue due to small amounts of ALT and AST present in the serum of culture medium. When a culture supernatant of a well treated with 5% ethyl alcohol was applied to the strip, the yellowish white zone was changed to dark blue, indicating rapidly increased AST and ALT activity. In contrast, when cells were dosed with ethyl alcohol to induce cell damage and then with the extract from L. caerulea fruits (0.3 mg/ml), the yellowish white zone changed to light blue. This result was also observed in the case in which the extract from L. caerulea fruits (0.3 mg/ml) was administered alone. Also, compared to the culture supernatant of the well treated with ethyl alcohol but not treated with the extract from L. caerulea fruits, the development of light blue indicates a decrease in ALT and AST levels.

### Example 6

### Toxicity Test

The hot water extract or hot water alcohol extract prepared in Example 1 was dissolved in distilled water, and administered to mice (ten per group) at a dosage of 500 mg/kg. Then, the mice were monitored for 7 days. No death was observed, indicating that the extract was not toxic.

### Example 7

### Evaluation of the Effect of the Extract from L. caerulea Fruits on Acute Hepatitis Induced in Mice

In order to determine whether the extract from L. caerulea fruits has the ability to restore liver function in acute hepatitis-induced mice, this test was carried out as follows. Forty mice (ICR) were divided into four groups A, B, C and D, each group consisting of ten mice. Group A was not dosed with any drug. Group B was allowed to ingest olive oil alone. Group C was orally dosed with 100 μl of silymarin (Sigma), which was dissolved in olive oil at 20 mg/ml, for 3 days. Group D was orally dosed with 100 μl of the extract from L. caerulea fruits prepared in Example 1, which was dissolved in distilled water at 500 mg/ml, for 3 days. During the period of drug administration, Groups A, B, C and D all received only water. Then, 100 g of 1% carbon tetrachloride in olive oil was intraperitoneally injected into mice of Groups B, C and D. After 18 hrs, blood samples were collected from the mice in Groups A, B, C and D. AST and ALT activities were determined in the mouse blood according to the same method as in Example 5, and mean AST and ALT activities were compared among groups. In Group A, which was not treated with carbon tetrachloride, mean levels of AST and ALT activities were 23 IU/L and 37 IU/L, respectively. In Group B, which was treated with carbon tetrachloride, mean AST and ALT levels were 1,023 IU/L and 1,129 IU/L, respectively, indicating that acute hepatitis was induced. In Group C, which was pretreated with silymarin, mean AST and ALT levels were 337 IU/L and 446 IU/L, respectively. In contrast, in Group D, which was pretreated with the extract from L. caerulea fruits, mean levels of AST and ALT activities were 107 IU/L and 130 IU/L, respectively. The mean AST and ALT levels in mice pretreated with the extract from L. caerulea fruits decreased by 230 IU/L and 207 IU/L, respectively, in comparison with those in mice pretreated with silymarin. With respect to AST and ALT levels, silymarin restored liver function by 63.8%, and the extract from L. caerulea fruits by 89.0%. These results indicate that the extract from L. caerulea fruits had ability to restore damaged liver function which was 25% better than that of silymarin.

### Example 8

### Comparison of the Results of Histochemical Analysis Between the Extract from L. caerulea Fruits and Silymarin in Acute Hepatitis-Induced Mice

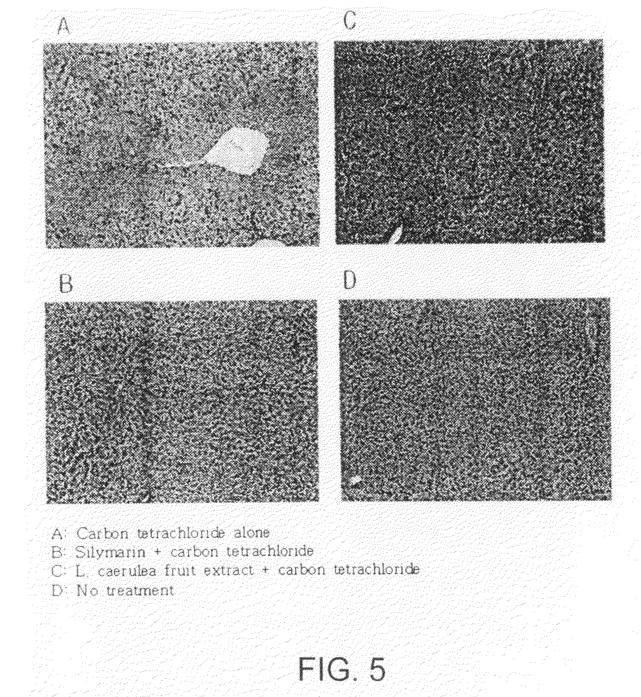

Histochemical analysis was performed with mouse liver tissues from Test Groups A, B, C and D in Example 7, whose mean AST and ALT levels were already determined in Example 7. The liver tissue from each mouse was sectioned, fixed in 10% formalin, immersed in different concentrations of ethyl alcohol to be dehydrated, and finally embedded in paraffin. The paraffin-embedded tissue was sectioned to a size of 4-5 μm. Each paraffin section was covered with a slide glass, stained with hematoxylin and eosin, and observed under an optical microscope (FIG. 5). Mice treated with carbon tetrachloride showed multiple scattered necrotic areas in the liver tissue, which were not stained with hematoxylin and eosin (FIG. 5, A panel). In liver tissues from mice pretreated with silymarin and mice pretreated with the extract from L. caerulea fruits (FIGS. 5, B and C panels), necrotic reaction rarely occurred compared to normal liver tissue (FIG. 5, D panel). In particular, when the liver tissue from mice pretreated with the extract from L. caerulea fruits was compared with that from mice pretreated with silymarin, fewer necrotic hepatocytes were found in the liver tissue from mice that received the extract from L. caerulea fruits.

### Example 9

### Clinical Test for the Liver Function-Restoring Effect of the Extract from L. caerulea Fruits

Three volunteers having hepatitis symptoms were orally dosed with the extract from L. caerulea fruits (1 g), prepared in Example 1, two times everyday for a period of 10 days or 20 days. Then, levels of the liver enzymes ALT and AST were measured. After the period of 10 days or 20 days, all of the three subjects showed a decrease in ALT and AST levels.

Subject 1: Male Age Forty

Before administration: suffered from hepatitis B since eight years previously

After administration: after 10-day administration, AST and ALT levels decreased from 32 to 11.1 and from 73 to 14.3, respectively; 20 days after administration, AST and ALT levels decreased to 10.3 and 10.6, respectively.

Subject 2: Male Age Thirty Five

Before administration: suffered from liver diseases and had high AST and AST levels at every physical examination

After administration: after 20-day administration, AST and ALT levels decreased from 53 to 2.25 and from 96 to 35, respectively; 60 days after administration, AST and ALT levels decreased to 2.25 and 5.75, respectively.

Subject 3: Female Age Thirty Five

Before administration: all family members suffered from hepatitis.

After administration: after 20-day administration, AST and ALT levels decreased from 69 to 26 and from 72 to 29, respectively.

### Example 10

### Preparation of Pharmaceutical Formulations

### Preparation Example 1

### Preparation of Powder Capsules

The 100 mg of the hot water extract prepared in Example 1 were mixed with 14.8 mg of lactose, 3 mg of crystalline cellulose, and 0.2 mg of magnesium stearate. The mixture was filled into a No. 5 capsule using a suitable device.

The components of powder capsules were summarized below.

Effective ingredient: 100 mg

Lactose: 14.8 mg

Crystalline cellulose: 3 mg

Magnesium stearate: 0.2 mg

### Preparation Example 2

### Preparation of Injectable Solution

An injectable solution containing 100 mg of the hot water extract prepared in Example 1 was prepared as follows.

100 mg of the hot water extract prepared in Example 1, 600 mg of sodium chloride and 100 mg of ascorbic acid were dissolved in distilled water, and the final volume was adjusted to 100 ml. The resulting solution was placed in a bottle and sterilized at 120° C. for 30 min.

The components of the injectable solution were as follows.

Effective ingredient: 1000 mg

Sodium chloride: 6000 mg

Ascorbic acid: 1000 mg

Distilled water: up to 1 L

### Preparation Example 3

### Preparation of Powders

Powders were prepared with the following composition according to the powder preparation method of the Korean Pharmacopeia.

1) Each Powder Contained

Effective ingredient (dry powder): 100 mg

Lactose: 100 mg

Talc: 10 mg

2) Each Powder Contained

Water-soluble fraction of effective ingredient: 100 mg

Lactose: 100 mg

Talc: 10 mg

### Preparation Example 4

### Preparation of Tablets

Tablets were prepared with the following composition according to the tablet preparation method of the Korean Pharmacopeia.

1) Each Tablet Contained

Effective ingredient (dry powder): 100 mg

Corn starch: 100 mg

Lactose: 100 mg

Magnesium stearate: 2 mg

2) Each Tablet Contained

Water-soluble fraction of effective ingredient: 100 mg

Corn starch: 100 mg

Lactose: 100 mg

Magnesium stearate: 2 mg

### Preparation Example 5

### Preparation of Capsules

Capsules were prepared with the following composition according to the capsule preparation method of the Korean Pharmacopeia.

1) Each Capsule Contained

Effective ingredient (dry powder): 100 mg

Corn starch: 100 mg

Lactose: 100 mg

Magnesium stearate: 2 mg

2) Each Capsule Contained

Water-soluble fraction of effective ingredient: 100 mg

Corn starch: 100 mg

Lactose: 100 mg

Magnesium stearate: 2 mg

### Preparation Example 6

### Preparation of Injections

Injections were prepared with the following composition according to the injectable preparation method of the Korean Pharmacopeia.

1) Each Injection (2 ml) Contained

Effective ingredient (dry powder): 50 mg

Sterile distilled water for injection: suitable amount

pH controller: suitable amount

2) Each Injection (2 ml) Contained

Water-soluble fraction of effective ingredient: 50 mg

Sterile distilled water for injection: suitable amount pH controller: suitable amount

### Preparation Example 7

### Preparation of Solutions

Solutions were prepared with the following composition according to the solution preparation method of the Korean Pharmacopeia.

1) Each Solution (100 ml) Contained

Effective ingredient (dry powder): 1 mg

Isomerized sugar: 10 g

Mannitol: 5 g

Purified water: suitable amount

2) Each Solution (100 ml) Contained

Water-soluble fraction of effective ingredient: 100 mg

Isomerized sugar: 10 g

Mannitol: 5 g

Purified water: suitable amount

### INDUSTRIAL APPLICABILITY

As described hereinbefore, the composition containing the L. caerulea extract according to the present invention has good ability to restore liver function and liver regeneration with no side effects such as toxicity. Thus, the present composition can be effectively used as a preventive and therapeutic agent for liver diseases.

1. A pharmaceutical composition having preventative and therapeutic effects on liver diseases, comprising an extract from Lonicera caerulea L. var. edulis.

2. The pharmaceutical composition according to claim 1, wherein the extract is obtained from fruits of Lonicera caerulea L. var. edulis.

3. The pharmaceutical composition according to claim 1, wherein the extract is prepared by an extraction process using water, an organic solvent, or a solvent mixture thereof.

4. The pharmaceutical composition according to claim 1, which has preventative and therapeutic effects on hepatitis, liver cirrhosis or fatty liver.

5. The pharmaceutical composition according to claim 1, wherein the extract from Lonicera caerulea L. var. edulis is present in an amount of 0.01% to 100% by weight based on the total weight of the composition.

6. The pharmaceutical composition according to claim 1, which is formulated into powders, granules, tablets, capsules or injections.
